(19) United States
(12) Patent Application Publication
(10) Pub. No.: US 2016/0331759 A1
(43) Pub. Date: Nov. 17, 2016

(54) Medicinal Composition for Treating Symptoms Related to Parkinson's Disease

(71) Applicant: Robert Rand, New York, NY (US)

(72) Inventor: Robert Rand, New York, NY (US)

(21) Appl. No.: 15/223,604

(22) Filed: Jul. 29, 2016

Related U.S. Application Data
(63) Continuation-in-part of application No. PCT/US15/20978, filed on Mar. 17, 2015.
(60) Provisional application No. 62/111,811, filed on Feb. 4, 2015.

(51) Int. Cl. A61K 31/55; A61K 47/12; A61K 31/51; A61K 31/525; A61K 31/375; A61K 31/197; A61K 31/4415; A61K 31/4188; A61K 31/519; A61K 31/714; A61K 47/38; A61K 31/455

(52) U.S. Cl. CPC A61K 31/55; A61K 47/38; A61K 47/12; A61K 31/51; A61K 31/525; A61K 31/455; A61K 31/197; A61K 31/4415; A61K 31/4188; A61K 31/519; A61K 31/714; A61K 31/375

(57) ABSTRACT

A medicinal composition is administered orally twice a day to a patient to ameliorate the symptoms related to Parkinson's disease. The medicinal composition includes mixing a quantity of ibogaine, a quantity of vitamin supplements and a quantity of manufacturing additives into a heterogeneous medicinal mixture. The quantity of ibogaine, formulated as ibogaine HCl, other non-toxic salts of ibogaine (an alkaloid of the family apocynaceae), or noribogaine, its active metabolite, is an active ingredient to regulate endogenous glial cell line-derived neurotrophic factor (GDNF). The quantity of vitamin supplements promotes the general health and neurological functions. The quantity of manufacturing additives bonds the quantity of ibogaine and the quantity of vitamin supplements together, as well as, provides anti-adherent properties to prevent the medicinal mixture from adhering to manufacturing equipment.

The current application claims priority to international PCT (Patent Cooperation Treaty) Application PCT/US 15/20978 filed on Mar. 17, 2015.

The international PCT Application PCT/US 15/20978 claims priority to the U.S. Provisional Patent application Ser. No. 62/111,811 filed on Feb. 4, 2015.

### FIELD OF THE INVENTION

The present invention relates generally to the treatment of Parkinson's disease. More specifically, the present invention relates particularly to the amelioration of motor and non-motor symptoms of the disease.

### BACKGROUND OF THE INVENTION

Parkinson's disease (PD) is a common adult-onset neurodegenerative disorder. Briefly described, PD is the second most common neurodegenerative disease after Alzheimer's disease which manifests during the 5th or 6th decade of human life. Clinically, PD is characterized by abnormal motor manifestations referred to as tremor [shaking], rigidity [stiffness], akinesia [paucity of spontaneous movements], and postural instability [poor balance] (TRAP). These cardinal features are due mainly, though not exclusively, to the loss of brain cells called dopaminergic neurons which are located in a discrete region of the brain known as the substantia nigra pars compacta (SNPC). PD is essentially a sporadic condition, meaning that it occurs, approximately 90% of the time, in absence of any genetic linkage; the remaining cases are inherited and caused by a variety of genetic defects. Yet, even in those rare genetic cases where we know the cause, the mechanism by which the loss of the dopamine neurons occurs in PD remains enigmatic. The consensus in the field is to consider that, without a more precise understanding of the neurobiology of PD, it will be nearly impossible to find an effective protective treatment for PD.

Recent published reports have shown that trophic factor supplements such as glial cell line-derived neurotrophic factor (GDNF) could mitigate dopaminergic neuronal loss both in experimental models and in PD patients. However, despite encouraging first set of results, subsequent clinical trials have failed to reproduce the reported beneficial effects. One problem with exogenous supplements of GDNF is that, even when delivered in a specific region, GDNF may diffuse away the site of interest and not necessarily hit the correct targets. To circumvent such a critical issue, a more promising strategy could be to force the glial cells [brain support cells] and neurons, which, in the brain, are normally responsible for GDNF production, to produce more GDNF. While such effect could be achieved by gene therapy through the use of using viral vectors, oral administration of drugs that supposedly stimulate GDNF production may be, at least for now, a safer and more convenient means to enhance GDNF production in the brain.

Ibogaine is a ritually used plant derivative (from the plant Tabernanthe Iboga) with “mind-expanding” qualities. Several scientific studies have shown ibogaine to exhibit anti-monoamine oxidase, anti-addictive, anti-epileptic, anti-depressive, and stimulant qualities. It has also been shown recently that the up-regulation of the GDNF pathway in the midbrain by Ibogaine is the molecular mechanism of its anti-addiction action and that this effect remains long after Ibogaine has disappeared from the system. This long-lasting effect is thought to be due primarily to the long-lasting induction of the GDNF pathway in which secreted GDNF induces its own expression leading to somewhat of a constant elevation of GDNF.

### BRIEF DESCRIPTION OF THE DRAWINGS

FIG. 1 is a block diagram detailing the ingredients of the present invention.

FIG. 2 is a flow diagram for the method of administering the present invention.

### DETAIL DESCRIPTIONS OF THE INVENTION

All illustrations of the drawings are for the purpose of describing selected versions of the present invention and are not intended to limit the scope of the present invention.

The present invention is a medicinal composition for treating symptoms related to parkinson's disease. The present invention ameliorates or eliminate the symptoms of Parkinson's disease through pharmacological means. Symptoms affected by the present invention include increased salivation, difficulty swallowing, loss of tongue and facial motor functions, decline of fine motor skills, balance, cognition, and feelings of temporary paralysis. A pharmacological means of regulating endogenous glial cell line-derived neurotrophic factor (GDNF) could improve safety and delivery issues. GDNF signaling is reportedly diminished by drugs of abuse. One such compound for regulating GDNF is the indole alkaloid, ibogaine. The ability of ibogaine to treat drug addiction and withdrawal has been anecdotally reported and verified in animal models of opiate, stimulant, and alcohol abuse.

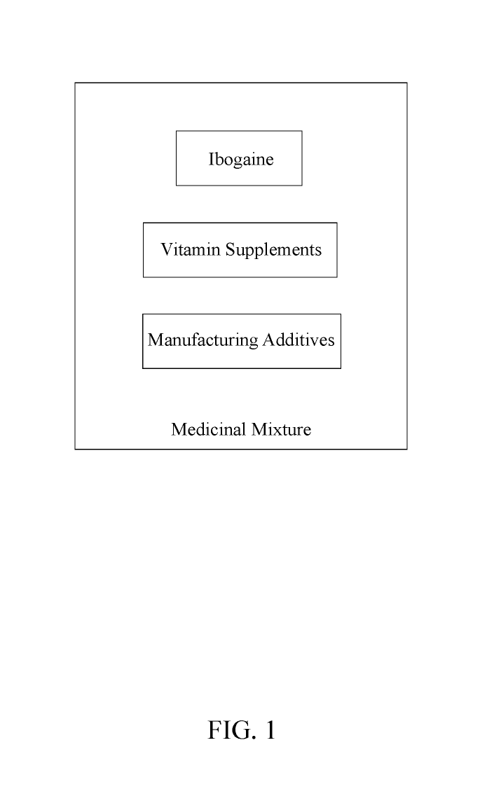

In accordance to FIG. 1, the present invention comprises a quantity of ibogaine, a quantity of vitamin supplements, and a quantity of manufacturing additives. The quantity of ibogaine assists in regulating GDNF by the attenuation of addiction. Ibogaine promotes long-lasting upregulation of GDNF expression, secretion and activation of downstream signaling pathways, and that these actions mediate the anti-addiction properties of this alkaloid. Ibogaine further exhibits anti-monoamine oxidase, anti-epileptic, anti-depressive and stimulant qualities. The ibogaine may be formulated as ibogaine hydrochloride (HCl), other non-toxic salts of ibogaine (an alkaloid of the family apocynaceae), or noribogaine, its active metabolite, for administration. In accordance to the preferred embodiment of the present invention, the quantity of ibogaine is formulated as ibogaine HCl such that the quantity of ibogaine is able to be ingested and metabolized by the patient. In accordance to other embodiments of the present invention, the quantity of ibogaine is formulated as 12-methyoxyibogaine such that the quantity of ibogaine is able to similarly be ingested and metabolized by the patient. The quantity of vitamin supplements provides additional compounds to promote general health for a patient. The quantity of manufacturing additives assists in combining the quantity of ibogaine and the quantity of vitamin supplements into a form which is able to be administered to the patient. The quantity of ibogaine, the quantity of manufacturing additives, and the quantity of vitamin supplements are heterogeneously combined into a medicinal mixture. The medicinal mixture is administered to a patient to be metabolized in order to regulate endogenous GDNF. The quantity of ibogaine is a range between 1% by weight (wt %) and 3 wt % of the medicinal mixture, as shown in Table 1. In accordance to the preferred embodiment, the quantity of ibogaine is preferred to be 1 wt % of the medicinal mixture. Thus, the preferred embodiment provides the quantity of ibogaine in a sufficient amount to ameliorate the symptoms related to Parkinson's disease.

TABLE 1
Medicinal Mixture

| Component | Approximate percent by weight (wt %) |
|---|---|
| Ibogaine | 1-3% |
| Vitamin Supplements | |
| Vitamin B1 | 1% |
| Vitamin B2 | 1% |
| Vitamin B3 | 2.6% |
| Vitamin B5 | 25.5% |
| Vitamin B6 | 1% |
| Vitamin B7 | 0.00052% |
| Vitamin B9 | 0.00052% |
| Vitamin B12 | 0.00001% |
| Vitamin C | 25.5% |
| Manufacturing Additives | |
| Microcrystalline Cellulose 101 | 38.5% |
| Magnesium Stearate | 1.5% |

In accordance to the preferred embodiment of the present invention, the quantity of vitamin supplements is selected from a group consisting of: a quantity of vitamin B1, a quantity of vitamin B2, a quantity of vitamin B3, a quantity of vitamin B5, a quantity of vitamin B6, a quantity of vitamin B7, a quantity of vitamin B9, a quantity of vitamin B12, a quantity of vitamin C, and combinations thereof. These compounds are also respectively known as thiamine, riboflavin, niacin, pantothenic acid, pyridoxine, biotin, folic acid, cobalamins, and ascorbic acid. The aforementioned compounds are cofactors for key metabolic processes or precursors needed for metabolic processes. Vitamin B1 promotes the generation of energy from carbohydrates and is involved with nerve function. Vitamin B2 promotes the activation of other vitamins. Vitamin B3 plays an important role in energy transfer reactions in the metabolism of glucose, fat and alcohol. Vitamin B5 is involved in the oxidation of fatty acids and carbohydrates. Vitamin B6 serves as a cofactor to many enzyme reactions, mainly in amino acid metabolism including biosynthesis of neurotransmitters. Vitamin B7 is involved in the metabolism of energy, amino acids and cholesterol. Vitamin B9 is involved in the transfer of single-carbon units in the metabolism of nucleic acids and amino acids and aids in the production of red blood cells. Vitamin B12 is essential in the production of blood cells in bone marrow, and for nerve sheaths and proteins.

In accordance to Table 1, the quantity of vitamin supplements of the preferred embodiment of the present invention is a combination of the quantity of vitamin B1, the quantity of vitamin B2, the quantity of vitamin B3, the quantity of vitamin B5, the quantity of vitamin B6, the quantity of vitamin B7, the quantity of B9, the quantity of vitamin B12, and the quantity of vitamin C. This combination is proportioned with the following weight percentages: the quantity of vitamin B1 is approximately 1 wt % of the medicinal mixture; the quantity of vitamin B2 is approximately 1 wt % of the medicinal mixture; the quantity of vitamin B3 is approximately 2.6 wt % of the medicinal mixture; the quantity of vitamin B5 is approximately 25.5 wt % of the medicinal mixture; the quantity of vitamin B6 is approximately 1 wt % of the medicinal mixture; the quantity of B7 is approximately 0.00052 wt % of the medicinal mixture; the quantity of B9 is approximately 0.00052 wt % of the medicinal mixture; the quantity of vitamin B12 is approximately 0.00001 wt % of the medicinal mixture; and the quantity of vitamin C is approximately 25.5 wt % of the medicinal mixture. The contribution for each of the quantity of vitamin supplements to the total mass of the medicinal mixture is intended to be sufficient to impart the beneficiary effects for each of the vitamin supplements compounds to the patient.

Further in accordance to the preferred embodiment of the present invention, the quantity of manufacturing additives is selected from a group consisting of: a quantity of microcrystalline cellulose 101 (MCC101), a quantity of magnesium stearate, and combinations thereof, as shown in Table 1. The quantity of MCC101 binds the quantity of ibogaine and the quantity of vitamin supplements together such that the medicinal mixture is able to be shaped into orally administrable form. Magnesium stearate is an anti-adherent which prevents the medicinal mixture from sticking to manufacturing equipment. In accordance to the preferred embodiment of the present invention, the quantity of manufacturing additives is a combination of MCC101 and magnesium stearate. This combination is proportioned with the quantity of MCC101 is approximately 38.5% of the medicinal mixture, such that the quantity of MCC101 is present in sufficient amount in order to bind the quantity of ibogaine and the quantity of vitamin supplements within the medicinal mixture, and the quantity of magnesium stearate is approximately 1.5% of the medicinal mixture, such that the quantity of magnesium stearate is present in sufficient amount to impart anti-adherent properties to the medicinal mixture.

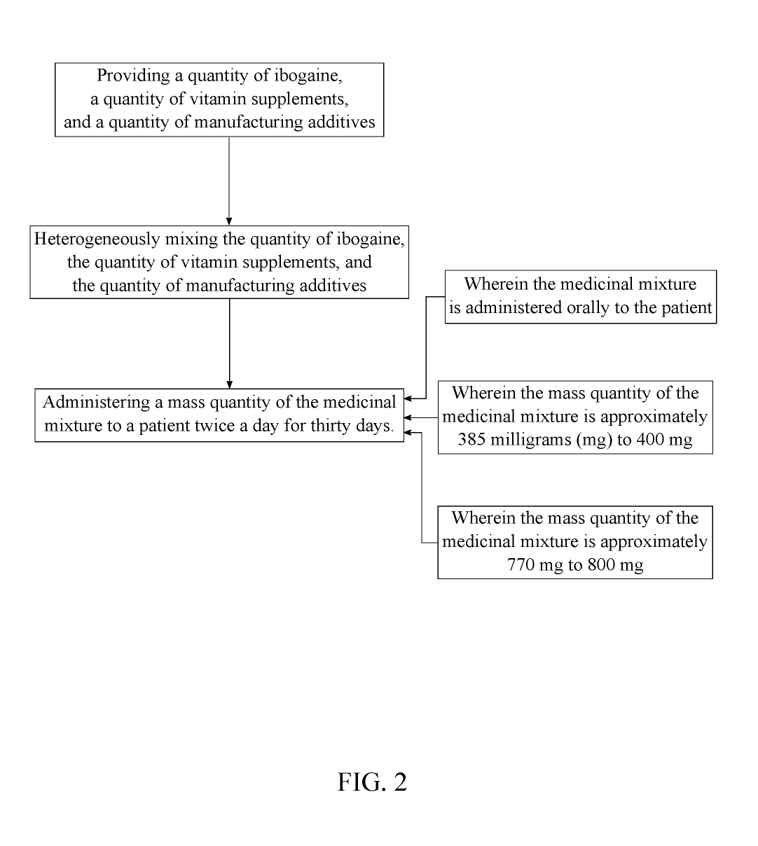

A method for administering the medicinal composition for treating the symptoms related to Parkinson's disease, in accordance to FIG. 2, comprises the steps of administering a mass quantity of the medicinal mixture to a patient twice a day for thirty days. The medicinal mixture is preferred to be administered orally to the patient such that the present invention is able to be metabolized by the patient. In some embodiments for the method of administration of the present invention, the mass quantity of the medicinal mixture is approximately 385 milligrams (mg) to 400 mg. Alternatively, the patient receives twice the mass quantity of the present invention twice a day. The mass quantity of the medicinal mixture is approximately 770 mg to 800 mg. The mass quantity of the medicinal mixture provides sufficient amounts for the quantity of ibogaine, the quantity of vitamin supplements, and the manufacturing additives to be effective.

Although the invention has been explained in relation to its preferred embodiment, it is to be understood that many other possible modifications and variations can be made without departing from the spirit and scope of the invention as hereinafter claimed.

What is claimed is:

1. A medicinal composition for treating symptoms related to Parkinson's disease comprises:
a quantity of ibogaine;
a quantity of vitamin supplements;
a quantity of manufacturing additives; and
the quantity of ibogaine, the quantity of manufacturing additives and the quantity of vitamin supplements being heterogeneously combined into a medicinal mixture.

2. The medicinal composition for treating symptoms related to Parkinson's disease, as claimed in claim 1, comprises:
the quantity of ibogaine being formulated as ibogaine hydrochloride.

3. The medicinal composition for treating symptoms related to Parkinson's disease, as claimed in claim 1, comprises:
the quantity of ibogaine being formulated as 12-methyoxyibogaine.

4. The medicinal composition for treating symptoms related to Parkinson's disease, as claimed in claim 1, comprises:
the quantity of ibogaine being a range between 1% by weight (wt %) and 3 wt % of the medicinal mixture.

5. The medicinal composition for treating symptoms related to Parkinson's disease, as claimed in claim 4, comprises:
the quantity of ibogaine being approximately 1 wt % of the medicinal mixture.

6. The medicinal composition for treating symptoms related to Parkinson's disease, as claimed in claim 1, comprises:
the quantity of vitamin supplements being selected from a group consisting of: a quantity of vitamin B1, a quantity of vitamin B2, a quantity of vitamin B3, a quantity of vitamin B5, a quantity of vitamin B6, a quantity of vitamin B7, a quantity of vitamin B9, a quantity of vitamin B12, a quantity of vitamin C, a quantity of folic acid, and combinations thereof.

7. The medicinal composition for treating symptoms related to Parkinson's disease, as claimed in claim 6, comprises:
the quantity of vitamin supplements being a combination of: the quantity of vitamin B1, the quantity of vitamin B2, the quantity of vitamin B3, the quantity of vitamin B5, the quantity of vitamin B6, the quantity of vitamin B7, the quantity of B9, the quantity of vitamin B12, and the quantity of vitamin C;
the quantity of vitamin B1 being approximately 1 wt % of the medicinal mixture;
the quantity of vitamin B2 being approximately 1 wt % of the medicinal mixture;
the quantity of vitamin B3 being approximately 2.6 wt % of the medicinal mixture;
the quantity of vitamin B5 being approximately 25.5 wt % of the medicinal mixture;
the quantity of vitamin B6 being approximately 1 wt % of the medicinal mixture;
the quantity of vitamin B7 being approximately 0.00052 wt % of the medicinal mixture;
the quantity of vitamin B9 being approximately 0.00052 wt % of the medicinal mixture;
the quantity of vitamin B12 being approximately 0.00001 wt% of the medicinal mixture; and
the quantity of vitamin C being approximately 25.5 wt % of the medicinal mixture.

8. The medicinal composition for treating symptoms related to Parkinson's disease, as claimed in claim 1, comprises:
the quantity of manufacturing additives being selected from a group consisting of: a quantity of microcrystalline cellulose 101 (MCC101), a quantity of magnesium stearate, and combinations thereof.

9. The medicinal composition for treating symptoms related to Parkinson's disease, as claimed in claim 8, comprises:
the quantity of manufacturing additives being a combination of: the quantity of MCC101 and the quantity of magnesium stearate;
the quantity of MCC101 being approximately 38.5 wt % of the medicinal mixture; and
the quantity of magnesium stearate being approximately 1.5 wt % of the medicinal mixture.

10. A method of administering the medicinal composition for treating symptoms related to Parkinson's disease, as claimed in claim 1, comprises:
administering a mass quantity of the medicinal mixture to a patient twice a day for thirty days.

11. The method of administering the medicinal composition for treating symptoms related to Parkinson's disease, as claimed in claim 10, wherein the mass quantity of the medicinal mixture is approximately 385 milligrams (mg) to 400 mg.

12. The method of administering the medicinal composition for treating symptoms related to Parkinson's disease, as claimed in claim 10, wherein the mass quantity of the medicinal mixture is approximately 770 mg to 800 mg.

13. The method of administering the medicinal composition for treating symptoms related to Parkinson's disease, as claimed in claim 10, wherein the medicinal mixture is administered orally to the patient.
